(19) United States
(12) Patent Application Publication
(10) Pub. No.: US 2016/0310748 A1
(43) Pub. Date: Oct. 27, 2016

(54) SEALED SEPARATOR AND TAB INSULATOR FOR USE IN AN ELECTROCHEMICAL CELL

(71) Applicant: Medtronic, Inc., Minneapolis, MN (US)

(72) Inventor: Paul B. AAMODT, Minnetonka, MN (US)

(21) Appl. No.: 14/692,774

(22) Filed: Apr. 22, 2015

(51) Int. Cl. A61N 1/378; H01M 6/02; H01G 9/10; H01G 9/048; H01G 9/02; H01G 9/025; H01M 2/16; H01M 10/04

(52) U.S. Cl. CPC A61N 1/378; H01M 2/1673; H01M 6/02; H01M 10/0431; H01M 2/1686; H01M 2/1653; H01G 9/048; H01G 9/02; H01G 9/025; H01G 9/10; H01M 2220/00

(57) ABSTRACT

Electrodes having a sealed separator wherein the seal circumscribes the edges of the electrode and circumscribes a portion of the edges of the tab portion or tab portions of the electrode.

### FIELD

This disclosure relates to electrochemical cells, and more particularly to electrodes within an electrochemical cell having a sealed separator.

### BACKGROUND

Implantable medical devices (IMDs) detect and deliver therapy for a variety of medical conditions in patients. IMDs include implantable pulse generators (IPGs) or implantable cardioverter-defibrillators (ICDs) that deliver electrical stimuli to tissue of a patient. ICDs typically comprise, inter alia, a control module, a capacitor, and a battery that are housed in a hermetically sealed container or housing. When therapy is required by a patient, the control module signals the battery to charge the capacitor, which in turn discharges electrical stimuli to tissue of a patient.

The battery includes a case, a liner, an electrode assembly, and electrolyte. The liner insulates the electrode assembly from the case. The electrode assembly includes electrodes, an anode and a cathode, with a separator therebetween. For a flat plate battery, an anode comprises a set of anode electrode plates with a set of tabs extending therefrom. The set of tabs are electrically connected. Each anode electrode plate includes a current collector with anode material disposed thereon. A cathode is similarly constructed.

Electrolyte, introduced to the electrode assembly via a fill port in the case, is a medium that facilitates ionic transport and forms a conductive pathway between the anode and cathode. An electrochemical reaction between the electrodes and the electrolyte causes charge to be stored on the cathode.

### SUMMARY

Electrodes within sealed separators are disclosed. Electrodes disclosed in this disclosure include an electrode portion and a tab portion or tab portions. An electrode within a sealed separator includes an electrode between layers of a separator, the layers of separator bonded together forming a seal. The seal of the bonded separator layer circumscribes all of the edges of the electrode portion of the electrode and a portion of the edges of the tab portion(s).

In one embodiment, an electrochemical cell comprises a first substantially planar electrode, the first substantially planar electrode comprising a substantially planar electrode portion and a tab portion extending from the electrode portion and having tab edges that are parallel to one another; and first and second separator layers sandwiching the first substantially planar electrode, the first and second separator layers bonded together forming a seal, the seal circumscribing all edges of the electrode portion and a portion of the parallel edges of the tab portion.

In another embodiment, a medical device comprises a medical device housing, a control module within the housing, and an electrochemical cell within the housing, wherein the electrochemical cell comprises

a first substantially planar electrode, the first substantially planar electrode comprising a substantially planar electrode portion and a tab portion extending from the electrode portion and having tab edges that are parallel to one another; and
first and second separator layers sandwiching the first substantially planar electrode, the first and second separator layers bonded together forming a seal, the seal circumscribing all edges of the electrode portion and a portion of the parallel edges of the tab portion.

### BRIEF DESCRIPTION OF THE DRAWINGS

FIG. 1 is a depiction of an embodiment of an electrode within a sealed separator.

FIG. 2 is a depiction of a close-up of a tab portion after the separator was sealed.

FIGS. 3A and 3B are depictions of electrodes within a sealed separator suitable for use in a coiled electrochemical cell.

FIG. 4 is a depiction of a coiled electrochemical cell made using electrodes sealed within separators.

### DETAILED DESCRIPTION

An electrode within a sealed separator is disclosed. Electrodes disclosed include a current collector and an electrode material on the current collector. Such electrodes are substantially planar for use in a stacked plate or wound electrochemical cell, for example a battery or a capacitor. “Substantially planar” electrodes also include electrodes having two major surfaces and which may be solid in cross section, for example a solid lithium or tantalum electrode. Electrodes disclosed in this disclosure include an electrode portion and a tab portion or tab portions. An electrode within a sealed separator includes an electrode between layers of a separator, the layers of separator bonded together forming a seal. The seal of the bonded separator layer circumscribes all of the edges of the electrode portion of the electrode and a portion of the edges of the tab portion(s). The separator bonded to and around a portion of the tab portion insulates and protects the tab portion. The electrodes described in the application are useful in primary and secondary batteries and in capacitors.

The separator is bonded together such that a substantial portion of the tab portion(s) is sealed within the separator, with a portion of the tab portion(s) uncovered by separator. The seal bonding the separator layers together is typically formed by the application of heat and pressure. Any excess separator material is trimmed away, desirably when the separator layers are sealed. The heat and pressure also seals the ends or edges of the separator material onto or across the major surfaces of the tab portion(s).

Either the negative or positive electrode may be sealed in the manner described above. In typical applications, the positive electrode or cathode electrode is sealed within separator.

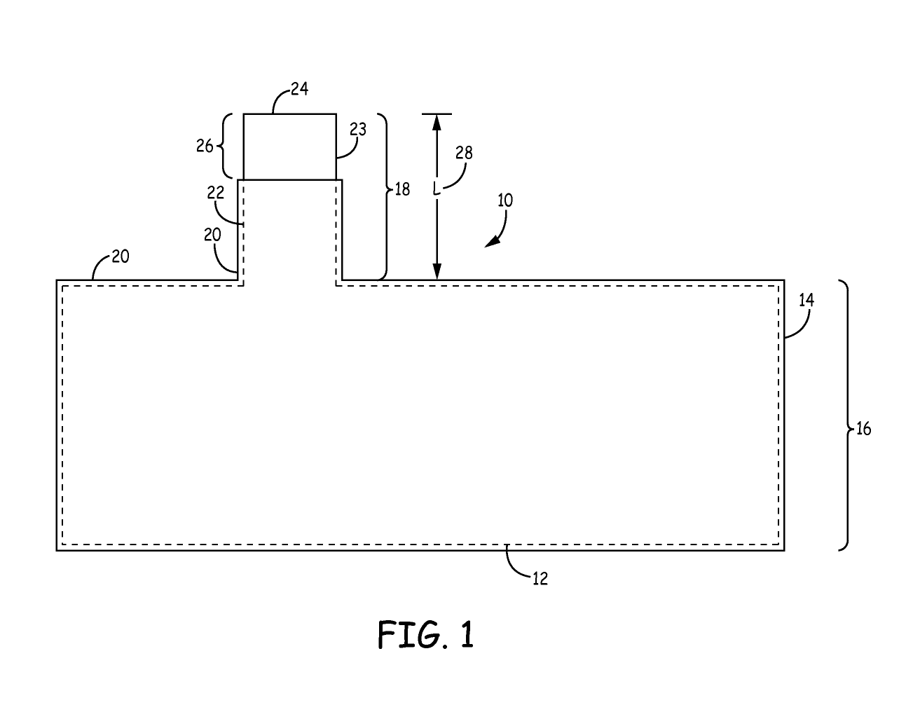

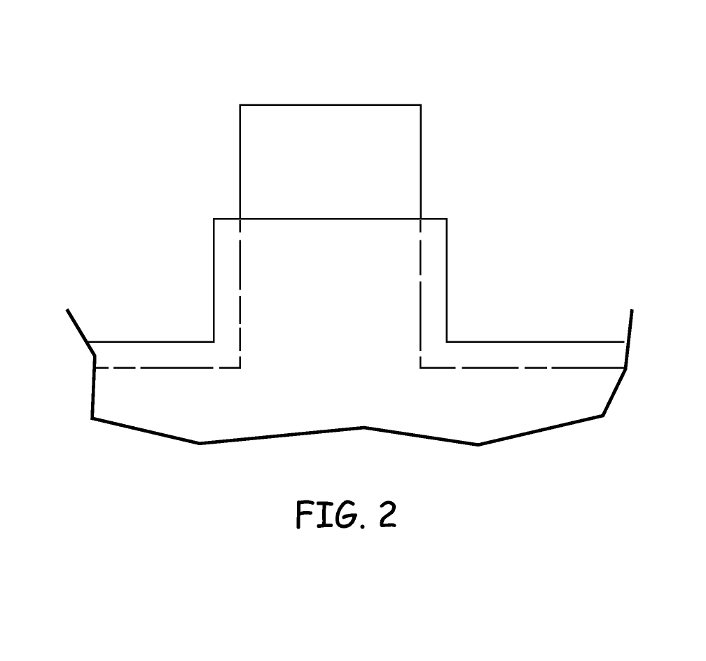

One embodiment of an electrode within a sealed separator is depicted in FIG. 1. Sealed electrode 10 includes an electrode 12 shown by dashed lines and a separator 14 substantially encasing the electrode. Electrode 12 includes an electrode portion 16 and a tab portion 18. Tab portion 18 has first and second parallel edges 22, 23 and a third edge 24 in between and connecting first and second parallel edges of tab portion. Separator seal 20 circumscribes or follows the outline of the edges of the electrode portion. Separator seal 20 also circumscribes or follows a portion of the parallel edges 22, 23 of tab portion, leaving an uncovered portion 26 of the tab portion. FIG. 2 shows a close up of a tab portion of an electrode after the separator layers have been bonded together to form a seal.

The parallel edges of the tab portion have a length, “L” 28. The area or amount of the tab portion sealed within separator may be described as a percentage of the length of the parallel edges of the tab portion are circumscribed by a separator seal. In one example, the seal of the separator extends along at least 30 percent of the length of the parallel edges of the tab portion. In other examples, the seal of the separator extends along from 30 percent to 95 percent of the length of the parallel edges of the tab portion, and can extend along any range or point within this range.

The amount of the tab portion left uncovered also depends upon whether the electrochemical cell is configured as a stacked plate or a coiled or wound cell. For example, in a stacked plate electrochemical cell, spacers are typically placed between the tabs of adjacent plates, and are welded together. In such a design, the area of the tab portion sealed within separator tends to be a smaller percentage in order to avoid thermal damage. In a coiled or wound electrode configuration, a larger area of the tab portion is sealed within separator as spacers are typically not used and other attachment means that generate less heat can be used, for example, resistance spot welding. In most applications, some portion of the tab portion area is left un-sealed by separator as an attachment or weld zone.

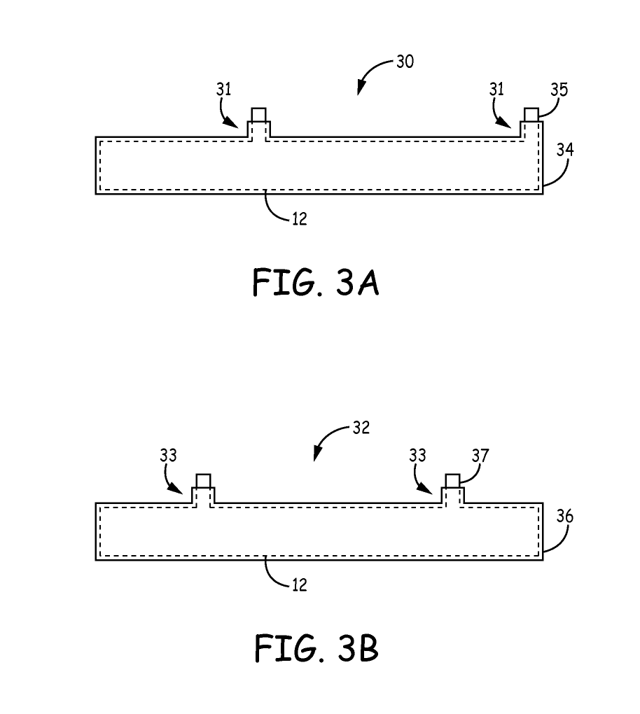

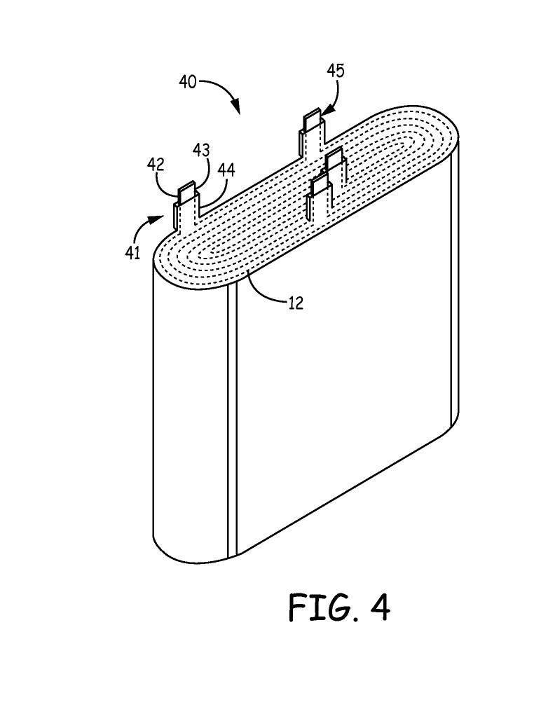

FIGS. 3A and 3B show another embodiment of electrodes 12 shown by dashed lines within a sealed separator 34, 36, 44. Sealed electrodes 30 and 32 have a configuration suited for being wound around a mandrel after being stacked to form a wound electrochemical cell 40 as depicted in FIG. 4. Separator seal 34, 36 and 44 also circumscribed or follows a portion of the parallel edges, for example 42 and 43 of tab portions 31, 33 and 41 in FIGS. 3A, 3B and 4, leaving uncovered portions 35, 37 and 45.

Although the tab portions in the above embodiments have been depicted and described as substantially planar and substantially rectangular in shape, the tab portions could be cylindrical, cuboid, or triangular in shape and can have a thickness that is less than , equal to, or greater than the thickness of the electrode portion.

Each electrode plate includes a current collector or grid, a tab extending therefrom, and electrode material. Tab and current collector comprises conductive material (e.g. copper, aluminum, titanium, nickel, platinum, etc.).

Negative electrode or anode material includes elements from Group IA, IIA or IIIB of the periodic table of elements (e.g. lithium, sodium, potassium, etc.), alloys thereof, intermetallic compounds (e.g. Li—Si, Li—B, Li—Si—B etc.), or an alkali metal (e.g. lithium, etc.) in metallic form.

Positive electrode or cathode materials includes metal oxides (e.g. vanadium oxide, silver vanadium oxide (SVO), manganese dioxide, tantalum oxide etc.), carbon monofluoride and hybrids thereof (e.g., CFₓ+MnO₂, CFx+SVO), combination silver vanadium oxide (CSVO), lithium ion, other rechargeable chemistries, or other suitable compounds.

Typically, the separator comprises a polymeric material that is porous or microporous. The separator material may itself be made of multiple layers of polymeric materials that are bonded together. Useful separators have a thickness in a range of from 0.0008 to 0.0035 in (0.02 to 0.09 mm), and may be any thickness or range of thicknesses in between such range. Typical materials used in separators include polyethylene, polypropylene and polytetrafluoroethylene. Commercially available separator material is available under the brandname “CELGARD” from Celgard, LLC, Charlotte, N.C.

The electrochemical cells described in this application typically contain an electrolyte. Useful electrolytes are nonaqueous

[symbol]

In general, electrodes sealed in a separator as described in this application are made by placing and electrode between two layers of separator material and applying an appropriate amount of heat and pressure just outside the edges of the electrode portion and tab portion, forming a seal, and then trimming any excess separator material. In this manner, the separator layers covering the electrode portion and the tab portion are unitary, that is one piece. In other words, the separator sealing a portion of the tab portion is not a separate piece or pieces of separator that is attached separately from the separator sealing the electrode portion of the electrode. Such process steps can be automated using for example, heated platens to seal the separator material together and form a seal that circumscribes the edges of the electrode and a portion of the tab portion. Excess separator material can then be trimmed using a die or other cutting means such as a laser.

Multiple electrodes sealed within a separator as described in this application may be stacked and placed within an electrochemical cell housing which is typically hermetically sealed. In another embodiment, a stack of electrodes sealed within a separator as described in this application can be wound around a mandrel for example, to form a wound electrochemical cell and placed within a housing. Housings for electrochemical cells are typically made of a metal for example, aluminum, titanium, stainless steel or can be made of a polymeric material. Sealing of the housing is typically done through welding if the housing is made of metal. In certain uses, the housing may be a “crimp-lock” housing where hermeticity is not required. The electrochemical cell housings may have one or more hermetically sealed feedthroughs.

When used in a stacked plate or coiled battery or capacitor design, the sealed separator over the tab potion insulates the electrodes between stacked tabs. In a coiled battery design, the sealed separator on the tab portion protects the electrode from any breach in the separator of an adjacent electrode.

Various examples have been described. These and other examples are within the scope of the following claims.

What is claimed is:

1. An electrochemical cell comprising:
a first substantially planar electrode, the first substantially planar electrode comprising a substantially planar electrode portion and a tab portion extending from the electrode portion and having tab edges that are parallel to one another; and
first and second separator layers sandwiching the first substantially planar electrode, the first and second separator layers bonded together forming a seal, the seal circumscribing all edges of the electrode portion and a portion of the parallel edges of the tab portion.

2. The electrochemical cell of claim 1, further comprising a second substantially planar electrode stacked on the first substantially planar electrode.

3. The electrochemical cell of claim 2, wherein the second substantially planar electrode comprises a substantially planar electrode portion and a substantially planar tab portion extending from the electrode portion and having tab edges that are parallel to one another; and
first and second separator layers sandwiching the second substantially planar electrode, the first and second separator layers bonded together forming a seal, the seal circumscribing all edges of the substantially planar electrode portion and a portion of the parallel edges of the tab portion of the second substantially planar electrode.

4. The electrochemical cell of claim 3, wherein the stack of electrodes is in the form of a wound electrode stack.

5. The electrochemical cell of claim 1, wherein the seal of the separator extends along from 30 percent to 95 percent of the length of the parallel edges of the tab portion.

6. The electrochemical cell of claim 1, wherein the seal of the separator extends along from 40 percent to 95 percent of the length of the parallel edges of the tab portion.

7. The electrochemical cell of claim 1, wherein the seal of the separator extends along from 50 percent to 95 percent of the length of the parallel edges of the tab portion.

8. The electrochemical cell of claim 1, wherein the seal of the separator extends along from 60 percent to 95 percent of the length of the parallel edges of the tab portion.

9. The electrochemical cell of claim 1, wherein the seal of the separator extends along from 70 percent to 95 percent of the length of the parallel edges of the tab portion.

10. The electrochemical cell of claim 1, wherein the seal of the separator extends along from 80 percent to 95 percent of the length of the parallel edges of the tab portion.

11. The electrochemical cell of claim 1 wherein the separator includes multiple layers.

12. The electrochemical cell of claim 1 wherein the separator comprises polypropylene or polyethylene or polytetrafluoroethylene.

13. A medical device comprising:
a medical device housing;
a control module within the housing; and
an electrochemical cell within the housing, wherein the electrochemical cell comprises
a first substantially planar electrode, the first substantially planar electrode comprising a substantially planar electrode portion and a tab portion extending from the electrode portion and having tab edges that are parallel to one another; and
first and second separator layers sandwiching the first substantially planar electrode, the first and second separator layers bonded together forming a seal, the seal circumscribing all edges of the electrode portion and a portion of the parallel edges of the tab portion.

14. The medical device of claim 13 wherein the electrochemical cell further comprises a second substantially planar electrode adjacent the first substantially planar electrode.

15. The medical device of claim 14 wherein the electrochemical cell is within an electrochemical cell housing.

16. The electrochemical cell of claim 3 wherein the electrochemical cell is within an electrochemical cell housing.

17. The electrochemical cell of claim 1 wherein the electrochemical cell is a battery.

18. The electrochemical cell of claim 1 wherein the electrochemical cell is a capacitor.
